(19) United States
(12) Patent Application Publication
(10) Pub. No.: US 2026/0306384 A1
(43) Pub. Date: Oct. 8, 2026

(54) HAIR TREATMENT COMPOSITION

(71) Applicant: SELORA HAIRCARE LLC, Northville, MI (US)

(72) Inventors: Selda Dauti, Northville, MI (US); Bora Kalaj, Farmington Hills, MI (US)

(21) Appl. No.: 18/996,622

(22) PCT Filed: Jul. 13, 2023
(86) PCT No.: PCT/US2023/027626

§ 371 Date: Jan. 17, 2025

Related U.S. Application Data
(60) Provisional application No. 63/390,689, filed on Jul. 20, 2022.

(51) Int. Cl. A61K 8/64; A61K 8/34; A61K 8/36; A61K 8/365; A61K 8/41; A61K 8/42; A61K 8/9789; A61K 8/9794; A61Q 5/04

(52) U.S. Cl. CPC A61K 8/64; A61K 8/34; A61K 8/342; A61K 8/345; A61K 8/36; A61K 8/365; A61K 8/416; A61K 8/42; A61K 8/9789; A61K 8/9794; A61Q 5/04

(57) ABSTRACT

The present teachings generally relate to a hair treatment composition for straightening and/or smoothing hair and a method of formulating the hair treatment composition. The hair treatment composition includes one or more plant-based extracts or botanical agents, one or more conditioning agents, one or more anti-microbial agents, one or more emulsifying agents, one or more emollients, one or more fragrance components, and one or more hair straightening complexes.

### CROSS-REFERENCE TO RELATED APPLICATIONS

This application claims priority to U.S. Provisional Application No. 63/390,689, filed Jul. 20, 2022.

### FIELD

The present teachings relate generally to a hair treatment composition for straightening and/or smoothing hair and a method of formulating the hair treatment composition.

### BACKGROUND

Hair straightening or relaxing has become increasingly popular to achieve various hair styles. Numerous products and treatments are available to straighten curly, wavy, and/or frizzy hair for use at home or in a salon setting. However, many kits, products, and compositions adversely affect the user's hair, typically leaving it in a dull, brittle, and/or otherwise damaged state.

There remains a need for a hair treatment composition for straightening and/or smoothing hair that protects the hair from damage and enhances the shine, softness, manageability, and hydration of the hair.

### SUMMARY

The present teachings meet one or more of the above needs by providing a hair treatment composition. The hair treatment composition may comprise: one or more plant-based extracts/botanical agents; one or more conditioning agents; one or more anti-microbial agents; one or more emulsifying agents; one or more emollients; one or more fragrance components; and one or more hair straightening complexes.

The one or more hair straightening complexes may include a mixture of polycarbamide proteins, glyoxylic acid, sericin, and guar hydroxypropyltrimonium.

The mixture of polycarbamide proteins, glyoxylic acid, sericin, and guar hydroxypropyltrimonium may be present in an amount of about in an amount of about 15.0% wt. to 35.0% wt.

The one or more plant-based extracts or botanical agents may include aloe vera leaf juice, Canadian willowherb mixture, saw palmetto fruit extract solution, and bisabolol.

The hair treatment composition may comprise: about 47% wt. to 53% wt. of the aloe vera leaf juice; about 2.0% wt. to 4.0% wt. of the Canadian willowherb mixture; about 0.5 wt % to 2.0 wt % of the saw palmetto fruit extract solution; and about 0.3% wt. to 1.0% wt. of the bisabolol.

The one or more conditioning agents may include sodium gluconate, cetrimonium chloride, glycerin, behentrimonium chloride, and a Astrocaryum murumuru seed butter and Orbignya speciosa kernel oil mixture.

The hair treatment composition may comprise: about 0.2% wt. to 0.8% wt. of the sodium gluconate; about 2.0% wt. to 4.0% wt. of the cetrimonium chloride; about 2.0% wt. to 4.0% wt. of the glycerin; about 2.0% wt. to 5.0% wt. of the behentrimonium chloride; and about 1.0% wt. to about 2.0% wt. of the Astrocaryum murumuru seed butter and Orbignya speciosa kernel oil mixture.

The one or more anti-microbial agents may include an iodopropynyl butylcarbamate and phenoxyethanol mixture, and potassium sorbate.

The hair treatment composition may comprise: about 0.8% wt. to 2.0% wt. of the anti-microbial agent mixture; and about 0.2% wt. to 1.0% wt. of the potassium sorbate.

The one or more emulsifying agents may include cetyl stearyl alcohol; and a mixture of polyquatermium-37, dicaprylyl carbonate, and lauryl glucoside.

The hair treatment composition may comprise: about 3.0% wt. to 5.0% wt. of the cetyl stearyl alcohol; and about 1.0% wt. to 3.5% wt. of the mixture of polyquatermium-37, dicaprylyl carbonate, and lauryl glucoside.

The one or more emollients may include MCT oil; prickly pear seed oil; soybean glycerides and butyrospermum parkii (shea) butter unsaponifiables mixture; a mixture of dilinoleic acid/propanediol copolymer, phytosterol sunflowerseedate, and hydrogenated vegetable glycerides; and dimer dilinoleyl dimer dilinoleate.

The hair treatment composition may comprise: about 0.5% wt. to 1.5% wt. of the MCT oil; about 0.07% wt. to 0.5% wt. of the prickly pear seed oil; about 0.500% wt. to 1.000% wt. of the soybean glycerides and butyrospermum parkii (shea) butter unsaponifiables mixture; about 0.2% wt. to 0.8% wt. of the dilinoleic acid/propanediol copolymer, phytosterol sunflowerseedate, and hydrogenated vegetable glycerides mixture; and about 0.2% wt. to 0.8% wt. of dimer dilinoleyl dimer dilinoleate.

The hair treatment composition may comprise one or more fragrance components; wherein the one or more fragrance components include a flower oil extract, a citrus oil extract, or both.

The hair treatment composition may straighten and/or smooths hair.

The hair treatment composition may be a gel, serum, cream, lotion, or spray.

The present teachings provide for a hair treatment composition that may address at least some of the needs identified above. The hair treatment composition may comprise: water, polycarbamide proteins, glyoxylicacid, cetearyl alcohol, gycerin, behentrimonium chloride, cetrimonium chloride, astrocaryum murumuru seed butter, orbignya speciosa kernel oil, sericin, guar hydroxypropyltrimonium chloride, epilobium angustifolium flower/leaf/stem extract, serenoa serrulata fruit extract, bisabolol, opuntia ficus-indica seed oil, dimer dilinoleyl dimer dilinoleate, soybean glycerides, butyrospermum parkii (shea) butter unsaponifiables, dilinoleic acid/propanediol copolymer, phytosterol sunflowerseedate, hydrogenated vegetable glycerides, caprylic/capric triglyceride, Lavandula angustifolia (lavender) oil, polyquaternium-37, dicaprylyl carbonate, lauryl glucoside, potassium sorbate, fragrance, iodopropynyl butylcarbamate, phenoxyethanol, and sodium gluconate.

The present teachings provide a method for formulating a hair treatment composition that may address at least some of the needs identified above. The method may comprise separately mixing a first part, separately mixing a second part, mixing the first and second parts together to form an emulsion, separately mixing a third part, mixing the third part into the mixed first and second parts.

The first part may comprise aloe vera leaf juice, Canadian willowherb mixture, saw palmetto fruit extract solution, bisabolol, sodium gluconate, cetrimonium chloride, glycerin, and an iodopropynyl butylcarbamate and phenoxyethanol mixture.

The second part may comprise behentrimonium chloride; a soybean glycerides and butyrospermum parkii (shea) butter unsaponifiables mixture; dimer dilinoleyl dimer dilinoleate; prickly pear seed oil; cetyl stearyl alcohol; an Astrocaryum murumuru seed butter and Orbignya speciosa kernel oil mixture a mixture of dilinoleic acid/propanediol copolymer, phytosterol sunflowerseedate, and hydrogenated vegetable glycerides; and MCT oil.

The third part may comprise one or more fragrance components; potassium sorbate; a mixture of polycarbamide proteins, glyoxylic acid, sericin, and guar hydroxypropyltrimonium; a mixture of polyquatermium-37, dicaprylyl carbonate, and lauryl glucoside.

The first and second parts may be heated to 75-80° C. prior to the mixing of the first and second parts.

The mixed first and second parts may be cooled before the mixing of the third part into the mixed first and second parts.

The third part may be heated to 45° C. for the separately mixing the third part.

The present teachings overcome the above concerns by providing a hair treatment composition for straightening and/or smoothing hair that protects the hair from severe damage and enhances the shine, softness, manageability, and hydration.

### DETAILED DESCRIPTION

As will be seen in further detail in the following description, in one aspect of the present teachings, there is envisioned a hair treatment composition. The hair treatment composition may be used for straightening and/or smoothing hair.

The hair treatment composition may include one or more plant-based extracts or botanical agents (e.g., plant derivatives, plant essences). The one or more plant-based extracts or botanical agents may function to aid in enhancing the overall straightening process; enhance the condition of the user's hair, scalp, skin, or any combination thereof; reduce the amount of irritation to the user's scalp; soothe the user's scalp; or any combination thereof.

Preferably, the one or more plant-based extracts or botanical agents may include aloe vera leaf juice. The aloe vera leaf juice may function as a soothing agent to provide moisture, soothing sensation, and/or cell regeneration properties to a user's hair, scalp, skin, or any combination thereof.

The aloe vera leaf juice may be a mixture. The mixture may comprise aloe vera leaf powder and water. The aloe vera leaf powder may be dissolved in the water. The aloe vera leaf juice may be comprised of about 15% wt. aloe vera leaf powder.

The aloe vera leaf powder may be derived from the Aloe barbadensis leaf. An example of a suitable aloe vera leaf powder is Innovaloe™ Aloe Vera Gel Spray Dried Powder, commercially available from AMB WELLNESS.

The hair treatment composition may comprise about 45% wt. or more, 47% wt. or more, or even 49% wt. or more of the aloe vera leaf juice. The hair treatment composition may comprise about 55% wt. or less, 53% wt. or less, or even 51% wt. or less of the aloe vera leaf juice. Preferably, the hair treatment composition may comprise about 47% wt. to 53% wt. of the aloe vera leaf juice.

Preferably, the one or more plant-based extracts or botanical agents may include Canadian willowherb (Epilobium angustifolium) extract. The Canadian willowherb extract may function as a non-steroidal anti-irritant ingredient to prevent and/or reduce skin redness induced by chemicals and/or UV exposure. The Canadian willowherb extract may be derived from the flower, leaf, and/or stem of Epilobium angustifolium.

The Canadian willowherb extract may be formulated into a mixture. The Canadian willowherb mixture may comprise the Canadian willowherb extract, water, and/or glycerin. For example, the Canadian willowherb mixture may comprise about 4.0% wt. to 6.0% wt. Canadian willowherb (Epilobium Angustifolium) extract, 55% wt. to 65% wt. glycerin, 30% wt. to 40% wt. water, and optionally 0.2% wt. to 0.4% wt. citric acid.

An example of a suitable Canadian willowherb mixture is Canadian Willow Herb™ 5C PF, commercially available from Lucas Meyer Cosmetics.

The hair treatment composition may comprise about 1.0% wt. or more, 1.5% wt. or more, 2.0% wt. or more, or even 2.5% wt. or more of the Canadian willowherb mixture. The hair treatment composition may comprise about 5.0% wt. or less, 4.5% wt. or less, 4.0% wt. or less, or even 3.5% wt. or less of the Canadian willowherb mixture. Preferably, the hair treatment composition may comprise about 2.0% wt. to about 4.0% wt. of the Canadian willowherb mixture.

Preferably, the one or more plant-based extracts or botanical agents may include saw palmetto (Serenoa serrulata) fruit extract. The saw palmetto fruit extract may function to provide moisturizing, conditioning, smoothing, and/or soothing properties to the user's hair, skin, scalp, or any combination thereof; as a humectant, to promote moisture retention by attracting water molecules from the surrounding environment; to prevent and/or treat hair loss; or any combination thereof.

The saw palmetto fruit extract may be formulated into a solution. The solution may comprise the saw palmetto fruit extract mixed with ethanol, water, glycerin, the like, or any combination thereof.

An example of a suitable saw palmetto fruit extract solution is GLB Saw Palmetto Extract, commercially available from Green Line-Andicor. Another example of a suitable saw palmetto fruit extract is Saw Palmetto Extract GNBN, commercially available from Provital.

The hair treatment composition may comprise about 0.2% wt. or more, 0.6% wt. or more, 0.8% wt. or more, or even 1.2% wt. or more of the saw palmetto fruit extract solution. The hair treatment composition may comprise about 3.0% wt. or less, 2.6% wt. or less, or even 2.2% wt. or less of the saw palmetto fruit extract solution. Preferably, the hair treatment composition may comprise about 0.5 wt % to about 2.0 wt % of the saw palmetto fruit extract solution.

Preferably, the one or more plant-based extracts or botanical agents may include bisabolol. The bisabolol may function to provide anti-inflammatory, anti-irritant, anti-bacterial, and/or non-allergic properties to the hair treatment composition. Bisabolol can be derived from at least Matricaria recutita and Myoporum crassiforlium, although other naturally-occurring sources may be contemplated by the present teachings.

The bisabolol may be in the alpha-bisabolol molecular structural form. An example of a suitable bisabolol is RTD™ Alpha Bisabolol Natural, commercially available from Hallstar.

The hair treatment composition may comprise about 0.1% wt. or more, 0.5% wt. or more, or even 0.9% wt. or more of the bisabolol. The hair treatment composition may comprise about 3.0% wt. or less, 2.6% wt. or less, or even 2.2% wt. or less of the bisabolol. Preferably, the hair treatment composition may comprise about 0.3% wt. to about 1.0% wt. of the bisabolol.

The hair treatment composition may comprise a blend of one or any combination of the aloe vera leaf juice, Canadian willowherb mixture, saw palmetto fruit extract solution, and bisabolol. Preferably the hair treatment composition may comprise the aloe vera leaf juice, Canadian willowherb mixture, saw palmetto fruit extract solution, and bisabolol.

The hair treatment composition may also include one or more conditioning agents to condition the user's hair, scalp, skin, or any combination thereof. The conditioning agent may function to aid in enhancing the feel, texture, and/or appearance of the user's hair, scalp, and/or skin; act as a lubricant on the skin's surface to give the skin a soft and smooth appearance; enhance the water content of the skin and/or hair; or any combination thereof.

Preferably, the one or more conditioning agents may include sodium gluconate. Sodium gluconate may function as a chelating agent (e.g., bind metallic ions to prevent the deterioration and/or discoloration of the hair treatment composition).

The hair treatment composition may comprise about 0.1% wt. or more, 0.3% wt. or more, or even 0.5% wt. or more of the sodium gluconate. The hair treatment composition may comprise about 1.0% wt. or less, 0.9% wt. or less, or even 0.7% wt. or less of the sodium gluconate. Preferably, the hair treatment composition may comprise about 0.2% wt. to about 0.8% wt. of the sodium gluconate.

Preferably, the one or more conditioning agents may include cetrimonium chloride. Cetrimonium chloride may function as a surfactant to lift dirt, oil, and debris from the user's hair, scalp, skin, or any combination thereof; to prevent and/or inhibit the buildup of static electricity to reduce static and/or “frizzy” hair (i.e., where individual hairs repel each other and project away from the main body of hair) during the straightening process; as an emulsifying agent to help prevent separation the components of the hair treatment composition; or any combination thereof.

An example of a suitable cetrimonium chloride is Dehyquart® A-CA, commercially available from BASF.

The hair treatment composition may comprise about 1.0% wt. or more, 1.5% wt. or more, 2.0% wt. or more, or even 2.5% wt. or more of the cetrimonium chloride. The hair treatment composition may comprise about 5.0% wt. or less, 4.5% wt. or less, 4.0% wt. or less, or even 3.5% wt. or less of the cetrimonium chloride. Preferably, the hair treatment composition may comprise about 2.0% wt. to about 4.0% wt. of the cetrimonium chloride.

Preferably, the one or more conditioning agents may include glycerin. Glycerin may function as a humectant to aid in the hair's moisture retention by attracting water molecules from the surrounding environment.

The hair treatment composition may comprise about 1.0% wt. or more, 1.5% wt. or more, 2.0% wt. or more, or even 2.5% wt. or more of the glycerin. The hair treatment composition may comprise about 5.0% wt. or less, 4.5% wt. or less, 4.0% wt. or less, or even 3.5% wt. or less of the glycerin. Preferably, the hair treatment composition may comprise about 2.0% wt. to about 4.0% wt. of the glycerin.

Preferably, the one or more conditioning agents may include behentrimonium chloride. The behentrimonium chloride may function to reduce the static of the user's hair.

An example of a suitable behentrimonium chloride is Incroquat™ Behenyl TMC-85, commercially available from Croda.

The hair treatment composition may comprise about 1.0% wt. or more, 1.5% wt. or more, 2.0% wt. or more, or even 2.5% wt. or more of the behentrimonium chloride. The hair treatment composition may comprise about 5.0% wt. or less, 4.5% wt. or less, 4.0% wt. or less, or even 3.5% wt. or less of the behentrimonium chloride. Preferably, the hair treatment composition may comprise about 2.0% wt. to about 5.0% wt. of the behentrimonium chloride.

Preferably, the one or more conditioning agents may include Astrocaryum murumuru seed butter, Orbignya speciosa kernel oil, or both. The Astrocaryum murumuru seed butter and Orbignya speciosa kernel oil may be provided as a mixture. The Astrocaryum murumuru seed butter and Orbignya speciosa kernel oil mixture may function as a natural alternative to silicone to provide visual improvement to the user's hair; to re-organize the cuticle; add shine and softness to the hair fiber; revitalize brightness to the user's hair; or any combination thereof.

An example of a suitable Astrocaryum murumuru seed butter and Orbignya speciosa kernel oil mixture is ActiveShine® Amazon 3R, commercially available from Chemyunion.

The hair treatment composition may comprise about 0.5% wt. or more, 1.0% wt. or more, or even 1.5% wt. or more of the Astrocaryum murumuru seed butter and/or Orbignya speciosa kernel oil. The hair treatment composition may comprise about 3.0% wt. or less, 2.5% wt. or less, or even 2.0% wt. or less of the Astrocaryum murumuru seed butter and/or Orbignya speciosa kernel oil. Preferably, the hair treatment composition may comprise about 1.0% wt. to about 2.0% wt. of the Astrocaryum murumuru seed butter and/or Orbignya speciosa kernel oil.

The hair treatment composition may comprise a blend of one or any combination of the sodium gluconate, cetrimonium chloride, glycerin, behentrimonium chloride, and a Astrocaryum murumuru seed butter and Orbignya speciosa kernel oil mixture. Preferably, the hair treatment composition may comprise the sodium gluconate, cetrimonium chloride, glycerin, behentrimonium chloride, and a Astrocaryum murumuru seed butter and Orbignya speciosa kernel oil mixture.

The hair treatment composition may include one or more anti-microbial agents. The one or more anti-microbial agents may function to provide antifungal and antibacterial properties to the hair treatment composition such as preserving the hair treatment composition from contamination by fungi, yeast, mold, bacteria, the like, or any combination thereof.

Preferably, the one or more anti-microbial agents may include a mixture of iodopropynyl butylcarbamate and phenoxyethanol. An example of a suitable iodopropynyl butylcarbamate and phenoxyethanol mixture is TroyCare™ FE02, commercially available from Troy Chemical Company.

The hair treatment composition may comprise about 0.5% wt. or more, 1.0% wt. or more, or even 1.5% wt. or more of the anti-microbial agent mixture. The hair treatment composition may comprise about 3.0% wt. or less, 2.5% wt. or less, or even 2.0% wt. or less of the anti-microbial agent mixture. Preferably, the hair treatment composition may comprise about 0.8% wt. to about 2.0% wt. of the anti-microbial agent mixture.

Preferably, the one or more anti-microbial agents may include potassium sorbate.

The hair treatment composition may comprise about 0.1% wt. or more, 0.3% wt. or more, 0.5% wt. or more, or even 0.7% wt. or more of the potassium sorbate. The hair treatment composition may comprise about 1.5% wt. or less, 1.3% wt. or less, 1.1% wt. or less, or even 0.9% wt. or less of the potassium sorbate. Preferably, the hair treatment composition may comprise about 0.2% wt. to about 1.0% wt. of the potassium sorbate.

The hair treatment composition may comprise a blend of one or any combination of a iodopropynyl butylcarbamate and phenoxyethanol mixture, and potassium sorbate. Preferably, the hair treatment composition may comprise a iodopropynyl butylcarbamate and phenoxyethanol mixture, and potassium sorbate.

The hair treatment composition may include one or more emulsifying agents. The one or more emulsifying agents may function to regulate the viscosity of the hair treatment composition; promote the blending of dissimilar compounds (e.g., normally immiscible liquids) within the hair treatment composition; aid in the stability of the hair treatment composition by preventing the individual ingredients from separating; or any combination thereof.

Preferably, the one or more emulsifying agents may include cetyl stearyl alcohol (also referred to as cetearyl alcohol or C16-18 alcohols). Ceteraryl alcohol may function to help smooth, soften, and de-frizz the user's hair. An example of a suitable cetyl stearyl alcohol is Lanette® O, commercially available from BASF.

The hair treatment composition may comprise about 2.0% wt. or more, 2.5% wt. or more, 3.0% wt. or more, or even 3.5% wt. or more of the cetyl stearyl alcohol. The hair treatment composition may comprise about 6.0% wt. or less, 5.5% wt. or less, 5.0% wt. or less, or even 4.5% wt. or less of the cetyl stearyl alcohol. Preferably, the hair treatment composition may comprise about 3.0% wt. to about 5.0% wt. of the cetyl stearyl alcohol.

Preferably, the one or more emulsifying agents may include a mixture of polyquatermium-37, dicaprylyl carbonate, and lauryl glucoside. The mixture of polyquatermium-37, dicaprylyl carbonate, and lauryl glucoside may function to offer enhanced sensory and conditioning properties to the user's hair. An example of a suitable mixture of polyquatermium-37, dicaprylyl carbonate, and lauryl glucoside is Cosmedia® Triple C, commercially available from BASF.

The hair treatment composition may comprise about 0.5% wt. or more, 1.0% wt. or more, 1.5% wt. or more, or even 2.0% wt. or more of the mixture of polyquatermium-37, dicaprylyl carbonate, and lauryl glucoside. The hair treatment composition may comprise about 4.5% wt. or less, 4.0% wt. or less, 3.5% wt. or less, or even 3.0% wt. or less of the mixture of polyquatermium-37, dicaprylyl carbonate, and lauryl glucoside. Preferably, of the hair treatment composition may comprise about 1.0% wt. to about 3.5% wt. of the mixture of polyquatermium-37, dicaprylyl carbonate, and lauryl glucoside.

The hair treatment composition may comprise a blend of one or any combination of cetyl stearyl alcohol; and a polyquatermium-37, dicaprylyl carbonate, and lauryl glucoside mixture. Preferably, the hair treatment composition may comprise cetyl stearyl alcohol; and a polyquatermium-37, dicaprylyl carbonate, and lauryl glucoside mixture.

The hair treatment composition may include one or more emollients. The one or more emollients may function to provide a protective barrier to the user's skin, hair, and/or scalp that contributes to moisturizing, softening, and smoothing of the same.

Preferably, the one or more emollients of the hair treatment composition may include MCT oil (caprylic/capric triglycerides). The MCT oil may function to slow the loss of water from the user's skin, scalp, and/or hair. An example of suitable MCT oil is MCT Oil, commercially available from Cambrian.

The hair treatment composition may comprise about 0.1% wt. or more, 0.3% wt. or more, 0.5% wt. or more, or even 0.7% wt. or more of the MCT oil. The hair treatment composition may comprise about 2.0% wt. or less, 1.7% wt. or less, 1.5% wt. or less, or even 1.3% wt. or less of the MCT oil. Preferably, the hair treatment composition may comprise about 0.5% wt. to about 1.5% wt. of the MCT oil.

Preferably, the one or more emollients of the hair treatment composition may include Opuntia ficus-indica seed oil (also known as prickly pear seed oil). The prickly pear seed oil may function to deeply condition hair due to the vitamin E contained in the oil and restore the natural shine of the user's hair due to the fatty acids and proteins contained in the oil, improving the overall health of the hair. An example of a suitable prickly pear seed oil is Prickly Pear, commercially available from New Directions Aromatics (NDA).

The hair treatment composition may comprise about 0.05% wt. or more, 0.06% wt. or more, or even 0.07% wt. or more of the prickly pear seed oil. The hair treatment composition may comprise about 1.0% wt. or less, 0.9% wt. or less, or even 0.8% wt. or less of the prickly pear seed oil. Preferably, the hair treatment composition may comprise about 0.07% wt. to about 0.5% wt. of the prickly pear seed oil.

Preferably, the one or more emollients of the hair treatment composition may include soybean glycerides and/or butyrospermum parkii (shea) butter unsaponifiables. The soybean glycerides and butyrospermum parkii (shea) butter unsaponifiables may be provided as a mixture. The soybean glycerides and butyrospermum parkii (shea) butter unsaponifiables mixture may function to provide a rich, creamy appearance to the hair treatment composition.

An example of a suitable soybean glycerides and butyrospermum parkii (shea) butter unsaponifiables mixture is Lipex® L′sens™, commercially available from AAK-Azelis.

The hair treatment composition may comprise about 0.250% wt. or more, 0.500% wt. or more, or even 0.750% wt. or more of the soybean glycerides and butyrospermum parkii (shea) butter unsaponifiables mixture. The hair treatment composition may comprise about 1.500% wt. or less, 1.250% wt. or less, or even 1.000% wt. or less of the soybean glycerides and butyrospermum parkii (shea) butter unsaponifiables mixture. Preferably, hair treatment composition may comprise about 0.500% wt. to about 1.000% wt. of the soybean glycerides and butyrospermum parkii (shea) butter unsaponifiables mixture.

Preferably, the one or more emollients of the hair treatment composition may include dilinoleic acid/propanediol copolymer, phytosterol sunflowerseedate, and/or hydrogenated vegetable glycerides. The dilinoleic acid/propanediol copolymer, phytosterol sunflowerseedate, and hydrogenated vegetable glycerides may be provided as a mixture. The mixture of dilinoleic acid/propanediol copolymer, phytosterol sunflowerseedate, and hydrogenated vegetable glycerides may function as a vegetable lanolin replacer, to provide shine and gloss to the user's hair.

An example of a suitable mixture of dilinoleic acid/propanediol copolymer, phytosterol sunflowerseedate, and hydrogenated vegetable glycerides is BIOSHINE 5000 S, commercially available from Grant.

The hair treatment composition may comprise about 0.1% wt. or more, 0.2% wt. or more, 0.3% wt. or more, or even 0.4% wt. or more of the dilinoleic acid/propanediol copolymer, phytosterol sunflowerseedate, and hydrogenated vegetable glycerides mixture. The hair treatment composition may comprise about 1.0% wt. or less, 0.9% wt. or less, 0.8% wt. or less, or even 0.7% wt. or less of the dilinoleic acid/propanediol copolymer, phytosterol sunflowerseedate, and hydrogenated vegetable glycerides mixture. Preferably, the hair treatment composition may comprise about 0.2% wt. to about 0.8% wt. of the dilinoleic acid/propanediol copolymer, phytosterol sunflowerseedate, and hydrogenated vegetable glycerides mixture.

Preferably, the one or more emollients of the hair treatment composition may include dimer dilinoleyl dimer dilinoleate. The dimer dilinoleyl dimer dilinoleate may function as a lanolin oil replacement to create a film on the user's hair to cancel out electrical charges with wet hair; and provide shine, hydration, softness, or a combination thereof to the user's hair. An example of a suitable dimer dilinoleyl dimer dilinoleate is LUSPLAN™ DD-DA7, commercially available from Kobo Products, Inc.

The hair treatment composition may comprise about 0.1% wt. or more, 0.2% wt. or more, 0.3% wt. or more, or even 0.4% wt. or more of dimer dilinoleyl dimer dilinoleate. The hair treatment composition may comprise about 1.0% wt. or less, 0.9% wt. or less, 0.8% wt. or less, or even 0.7% wt. or less of dimer dilinoleyl dimer dilinoleate. Preferably, the hair treatment composition may comprise about 0.2% wt. to about 0.8% wt. of dimer dilinoleyl dimer dilinoleate.

The hair treatment composition may include one or more fragrance components. The one or more fragrance components may function to mask the odor of other components within the hair treatment composition. The one or more fragrance components may function to provide an aesthetically pleasing smell to the hair treatment composition. The one or more fragrance components may be natural, synthetic, or any combination thereof.

Natural fragrance components may include those of vegetable, fruit, or animal origin, or a mixture thereof. For example, natural fragrance components may include oil extracts from flowers (lavender, rose, jasmine, etc.), fruits (citruses such as orange, lemon etc.), herbs (lemongrass, basil etc.), the like, or mixtures thereof.

The hair treatment composition may comprise about 0.1% wt. or more, 0.2% wt. or more, 0.3% wt. or more, or even 0.4% wt. or more of the overall fragrance component. The hair treatment composition may comprise about 1.0% wt. or less, 0.9% wt. or less, 0.8% wt. or less, or even 0.7% wt. or less of the overall fragrance component. Preferably, the hair treatment composition may comprise about 0.2% wt. to about 0.8% wt. of the overall fragrance component.

An example of a suitable fragrance component is Citrus Fragrance 110LQ211 Lot #446-A-4, commercially available from Sozio. The Citrus Fragrance 110LQ211 Lot #446-A-4 may be employed in the hair treatment composition in an amount of about 0.05% wt. to about 0.5% wt. Preferably, the Citrus Fragrance 110LQ211 Lot #446-A-4 may be present in an amount of about 0.07% wt. to about 0.3% wt. of the hair treatment composition.

An example of a suitable fragrance component is Lavender Population Essential Oil (Lavandula Angustifolia (Lavender) Oil), commercially available from New Directions Aromatics (NDA). The Lavender Population Essential Oil may be employed in the hair treatment composition in an amount of about 0.05% wt. to about 0.5% wt. Preferably, the Lavender Population Essential Oil may be present in an amount of about 0.1% wt. to about 0.3% wt. of the hair treatment composition.

The hair treatment composition may also include one or more straightening complexes. The straightening complexes may function to aid in enhancing the overall straightening and/or smoothing process and/or condition the user's hair, scalp, skin, or any combination thereof.

The one or more straightening complexes may include polycarbamide proteins, glyoxylic acid, sericin, guar hydroxypropyltrimonium, or any combination thereof. The polycarbamide proteins, glyoxylic acid, sericin, and guar hydroxypropyltrimonium may be provided as a mixture (e.g., with water).

An example of a suitable straightening complex is Seriliss, commercially available from Chemyunion. Seriliss may function to promote hair straightening and alignment of the user's hair; reduce and/or protect the user's hair from damage during the straightening process and reduce volume and curls; enhance shine, softness, manageability, and hydration of the user's hair; or any combination thereof. Seriliss may contain a mixture of water, polycarbamide proteins, glyoxylic acid, sericin, and guar hydroxypropyltrimonium.

The hair treatment composition may comprise about 15.0% wt. or more, 17% wt. or more, 19% wt. or more, or even 21% wt. or more of the polycarbamide proteins, glyoxylic acid, sericin, and guar hydroxypropyltrimonium mixture. The hair treatment composition may comprise about 35.0% wt. or less, 33.0% wt. or less, 31.0% wt. or less, or even 29.0% wt. or less of the polycarbamide proteins, glyoxylic acid, sericin, and guar hydroxypropyltrimonium mixture. Preferably, the hair treatment composition may comprise about 20.0 wt % to about 30.0 wt % of the polycarbamide proteins, glyoxylic acid, sericin, and guar hydroxypropyltrimonium mixture.

It is envisioned that any one of the above ingredients may also have secondary or tertiary functions. For example, the emollients of the hair treatment composition may also function as emulsifying agents, conditioning agents, etc. By further example, the plant-based extracts or botanical agents may also function as emollients, conditioning agents, fragrance components, etc. Further, it is envisioned that the hair treatment composition is a gel, serum, cream, lotion, or spray.

### Examples

The present teachings may be further explained by the following example. The following composition is given as an illustrative example and is not intended to limit the scope of the teachings herein. It should be understood that the amounts and ingredients within the example are not the only amounts and ingredients envisioned by the present teachings. The present teachings contemplate any combination of the ingredients and ranges of amounts as disclosed herein. The values presented in the illustrative example below are in weight-percent.

| | | |
|---|---|---|
| | | Weight |
| Ingredient | INCI | (kg)/% |

Part A

| | | |
|---|---|---|
| Aloe Vera Juice Extract | 15% solution of water and aloe | 51.700 |
| | barbadensis leaf powder | |
| Sodium Gluconate | Sodium gluconate | 0.200 |
| Dehyquart ® A-CA | Cetrimonium chloride (trimethyl | 2.400 |
| | hexadecyl ammonium chloride) | |
| Glycerin | Glycerin | 2.800 |
| Canadian Willowherb ™ | Glycerin, water, and Epilobium | 3.000 |
| 5C PF | angustifolium flower/leaf/stem | |
| | extract (CAS 289-821-5) | |
| Saw Palmetto Extract | Ethanol and Serenoa serrulata fruit | 1.000 |
| | extract (CAS 84604-15-9) | |
| Bisabolol Alpha Natural | Bisabolol | 0.500 |
| TroyCare ™ FE02 | 2% iodopropynyl butylcarbamate | 1.100 |
| | and 98% phenoxyethanol | |

Part B

| | | |
|---|---|---|
| Incroquat ™ | Behentrimonium chloride and | 2.500 |
| Behenyl TMC-85 | isopropyl alcohol | |
| Lipex ® L'sens ™ | Soybean glycerides and | 0.750 |
| | butyrospermum parkii (shea) butter | |
| | unsaponifiables | |
| LUSPLAN ™ DD-DA7 | Dimer dilinoleyl dimer dilinoleate | 0.250 |
| Prickly Pear | Opuntia ficus-indica seed oil | 0.100 |
| Lanette ® O | Cetyl stearyl alcohol | 4.000 |
| ActiveShine ® | Astrocaryum murumuru seed | 1.500 |
| Amazon 3R | butter, Orbignya speciosa kernel oil | |
| BIOSHINE 5000 S | Dilinoleic acid/propanediol | 0.300 |
| | copolymer, phytosteryl | |
| | sunflowerseedate, and | |
| | hydrogenated vegetable glycerides | |
| MCT Oil | Caprylic/capric triglyceride | 0.900 |

Part C

| | | |
|---|---|---|
| Citrus fragrance | Fragrance | 0.100 |
| 110LQ211 - | | |
| Lot#446-A-4 | | |
| Lavender | Lavandula angustifolia (Lavender) | 0.200 |
| population essential oil | Oil | |
| Potassium sorbate | Potassium sorbate | 0.300 |
| Seriliss | Water, polycarbamide proteins, | 24.500 |
| | glyoxylic acid, sericin, guar | |
| | hydroxypropyltrimonium | |
| Cosmedia ® Triple C | Polyquaternium-37, dicaprylyl | 1.900 |
| | carbonate, lauryl glucoside | |

| | |
|---|---|
| TOTAL | 100.000 |

The teachings herein also contemplate a method of forming the hair treatment composition. The formation of the hair treatment component may be achieved by any suitable method known by those of ordinary skill in the art. For instance, the method may include first separately weighing and mixing the components of the hair treatment composition of Part A and Part B, as listed above. The method may include separately heating the mixtures of Part A and Part B to 75-80° C. The method may include adding the mixture of Part B to Part A and emulsifying the mixture by constant mixture for 20 minutes or more. The method may further include allowing the resultant mixture of Part A and Part B to cool, while maintaining mixing.

The method may further include weighing and mixing the components of Part C, as listed above, at approximately 45° C. The method may include adding the Part C mixture to the resultant Part A/Part B mixture and homogenizing said overall mixture. Homogenization may take place by any suitable method known to those skilled in the art.

The resulting composition has a viscosity of about 4,500-5,000 cps, a solids content of about 24% and a pH of 1.40-1.70.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled.

The omission in the following claims of any aspect of subject matter that is disclosed herein is not a disclaimer of such subject matter, nor should it be regarded that the inventors did not consider such subject matter to be part of the disclosed inventive subject matter.

It will be appreciated that the above is by way of illustration only. Other ingredients may be employed in the composition disclosed herein, as desired, to achieve the desired resulting characteristics. Examples of other ingredients that may be employed include pH adjusters, osmolarity adjusters, and the like or any combination thereof.

The disclosures of all articles and references, including patent applications and publications, are incorporated by reference for all purposes.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. By use of the term “may” herein, it is intended that any described attributes that “may” be included are optional.

Unless otherwise stated, a teaching with the term “about” or “approximately” in combination with a numerical amount encompasses a teaching of the recited amount, as well as approximations of that recited amount. By way of example, a teaching of “about 100” encompasses a teaching of 100+/−15.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

Unless otherwise specified herein, quantities of each ingredient in the composition is expressed in percentage by weight (% wt.).

We claim:

1. A hair treatment composition comprising:
one or more plant-based extracts or botanical agents;
one or more conditioning agents;
one or more anti-microbial agents;
one or more emulsifying agents;
one or more emollients; and
one or more hair straightening complexes;
wherein the one or more hair straightening complexes includes a mixture of polycarbamide proteins, glyoxylic acid, sericin, and guar hydroxypropyltrimonium.

2. The hair treatment composition of claim 1, wherein the mixture of polycarbamide proteins, glyoxylic acid, sericin, and guar hydroxypropyltrimonium is present in an amount of about 15.0% wt. to 35.0% wt.

3. The hair treatment composition of claim 2, wherein the one or more plant-based extracts or botanical agents include aloe vera leaf juice, Canadian willowherb mixture, saw palmetto fruit extract solution, and bisabolol.

4. The hair treatment composition of claim 3, wherein the hair treatment composition comprises:
about 47% wt. to 53% wt. of the aloe vera leaf juice;
about 2.0% wt. to 4.0% wt. of the Canadian willowherb mixture;
about 0.5 wt % to 2.0 wt % of the saw palmetto fruit extract solution; and
about 0.3% wt. to 1.0% wt. of the bisabolol.

5. The hair treatment composition of claim 4, wherein the one or more conditioning agents include sodium gluconate, cetrimonium chloride, glycerin, behentrimonium chloride, and a Astrocaryum murumuru seed butter and Orbignya speciosa kernel oil mixture.

6. The hair treatment composition of claim 5, wherein the hair treatment composition comprises:
about 0.2% wt. to 0.8% wt. of the sodium gluconate;
about 2.0% wt. to 4.0% wt. of the cetrimonium chloride;
about 2.0% wt. to 4.0% wt. of the glycerin;
about 2.0% wt. to 5.0% wt. of the behentrimonium chloride; and
about 1.0% wt. to about 2.0% wt. of the Astrocaryum murumuru seed butter and Orbignya speciosa kernel oil mixture.

7. The hair treatment composition of claim 6, wherein the one or more anti-microbial agents include an iodopropynyl butylcarbamate and phenoxyethanol mixture, and potassium sorbate.

8. The hair treatment composition of claim 7, wherein the hair treatment composition comprises:
about 0.8% wt. to 2.0% wt. of the iodopropynyl butylcarbamate and phenoxyethanol mixture; and
about 0.2% wt. to 1.0% wt. of the potassium sorbate.

9. The hair treatment composition of claim 8, wherein the one or more emulsifying agents include cetyl stearyl alcohol; and a mixture of polyquatermium-37, dicaprylyl carbonate, and lauryl glucoside.

10. The hair treatment composition of claim 9, wherein the hair treatment composition comprises:
about 3.0% wt. to 5.0% wt. of the cetyl stearyl alcohol; and
about 1.0% wt. to 3.5% wt. of the mixture of polyquatermium-37, dicaprylyl carbonate, and lauryl glucoside.

11. The hair treatment composition of claim 10, wherein the one or more emollients include MCT oil; prickly pear seed oil; soybean glycerides and butyrospermum parkii (shea) butter unsaponifiables mixture; a mixture of dilinoleic acid/propanediol copolymer, phytosterol sunflowerseedate, and hydrogenated vegetable glycerides; and dimer dilinoleyl dimer dilinoleate.

12. The hair treatment composition of claim 11, wherein the hair treatment composition comprises:
about 0.5% wt. to 1.5% wt. of the MCT oil;
about 0.07% wt. to 0.5% wt. of the prickly pear seed oil;
about 0.500% wt. to 1.000% wt. of the soybean glycerides and butyrospermum parkii (shea) butter unsaponifiables mixture;
about 0.2% wt. to 0.8% wt. of the dilinoleic acid/propanediol copolymer, phytosterol sunflowerseedate, and hydrogenated vegetable glycerides mixture; and
about 0.2% wt. to 0.8% wt. of dimer dilinoleyl dimer dilinoleate.

13. The hair treatment composition of claim 12, wherein the hair treatment composition comprises one or more fragrance components; wherein the one or more fragrance components include a flower oil extract, a citrus oil extract, or both.

14. The hair treatment composition of claim 13, wherein the hair treatment composition straightens and/or smooths hair.

15. The hair treatment composition of claim 14, wherein the hair treatment composition is a gel, serum, cream, lotion, or spray.

16. A hair treatment composition comprising: water, polycarbamide proteins, glyoxylicacid, cetearyl alcohol, gycerin, behentrimonium chloride, cetrimonium chloride, astrocaryum murumuru seed butter, orbignya speciosa kernel oil, sericin, guar hydroxypropyltrimonium chloride, epilobium angustifolium flower/leaf/stem extract, serenoa serrulata fruit extract, bisabolol, opuntia ficus-indica seed oil, dimer dilinoleyl dimer dilinoleate, soybean glycerides, butyrospermum parkii (shea) butter unsaponifiables, dilinoleic acid/propanediol copolymer, phytosterol sunflowerseedate, hydrogenated vegetable glycerides, caprylic/capric triglyceride, Lavandula angustifolia (lavender) oil, polyquaternium-37, dicaprylyl carbonate, lauryl glucoside, potassium sorbate, fragrance, iodopropynyl butylcarbamate, phenoxyethanol, and sodium gluconate.

17. A method of formulating a hair treatment composition, the method comprising:
separately mixing a first part comprising:
aloe vera leaf juice,
Canadian willowherb mixture,
saw palmetto fruit extract solution,
bisabolol,
sodium gluconate,
cetrimonium chloride,
glycerin, and
an iodopropynyl butylcarbamate and phenoxyethanol mixture;
separately mixing a second part comprising:
behentrimonium chloride;
a soybean glycerides and butyrospermum parkii (shea) butter unsaponifiables mixture;
dimer dilinoleyl dimer dilinoleate;
prickly pear seed oil;
cetyl stearyl alcohol;
an Astrocaryum murumuru seed butter and Orbignya speciosa kernel oil mixture a mixture of dilinoleic acid/propanediol copolymer, phytosterol sunflowerseedate, and hydrogenated vegetable glycerides; and
MCT oil;
mixing the first and second parts together to form an emulsion;
separately mixing a third part comprising:
one or more fragrance components;
potassium sorbate;
a mixture of polycarbamide proteins, glyoxylic acid, sericin, and guar hydroxypropyltrimonium;
a mixture of polyquatermium-37, dicaprylyl carbonate, and lauryl glucoside; and
mixing the third part into the mixed first and second parts.

18. The method of claim 17, wherein the first and second parts are heated to 75-80° C. prior to the mixing of the first and second parts.

19. The method of claim 18, wherein the mixed first and second parts are cooled before the mixing of the third part into the mixed first and second parts.

20. The method of claim 19, wherein the third part is heated to 45° C. for the separately mixing the third part.
